(19) United States
(12) Patent Application Publication
(10) Pub. No.: US 2024/0274268 A1
(43) Pub. Date: Aug. 15, 2024

(54) DEVELOPING AND IMPLEMENTING PROGRAMS OF APPLIED BEHAVIORAL ANALYSIS

(71) Applicant: BehaviorSpan LLC, Aurora, CO (US)

(72) Inventors: J.J. Tomash, Aurora, CO (US); Seidy Snyder, Aurora, CO (US); Nicole Da Lima Leitao, Aurora, CO (US); Alex Eakins, Aurora, CO (US)

(21) Appl. No.: 18/414,283

(22) Filed: Jan. 16, 2024

Related U.S. Application Data
(60) Provisional application No. 63/479,957, filed on Jan. 13, 2023.

(51) Int. Cl. G16H 20/70; A61B 5/16; G16H 10/60

(52) U.S. Cl. CPC G16H 20/70; A61B 5/16; G16H 10/60

(57) ABSTRACT

A system and associated functionality are disclosed for developing and implementing programs of Applied Behavioral Analysis for individuals with developmental disabilities. The system (600) generally includes a network platform (604) that can be accessed by users (602) via a network (606). The illustrated platform (604) includes a subject database (610), a target database (612), and other elements, all operated by a processing system including one or more processors (608). The system (600) defines standardized targets, enables graphical interfaces for efficient development of programs based on standardized targets, and enables previously impossible statistical analysis of collected data.

### CROSS-REFERENCE TO RELATED APPLICATION

This application claims priority to U.S. Provisional Patent Application No. 63/479,957 entitled “Developing and Implementing Programs of Applied Behavioral Analysis,” filed Jan. 13, 2023, the contents of which are incorporated herein as if set forth in full and priority is claimed to the full extent allowable under U.S. law and regulations.

### FIELD OF THE INVENTION

The present invention relates generally to tools for use in developing and implementing programs of Applied Behavioral Analysis for individuals having developmental disabilities and, in particular, to such tools that define standardized targets, assist in target selection, enable graphical interfaces for efficient development of programs based on standardized targets, and enable improved statistical analysis of collected data.

### BACKGROUND OF THE INVENTION

### A. Overview of the Field

Applied Behavior Analysis (ABA) is a relatively new field compared to other treatments. It is a highly evidence-based approach to treating Autism and other developmental disabilities. ABA is a highly researched and proven treatment for Autism and other developmental disabilities, and hence it has exploded as an industry in the last decade. Such treatments are often initiated during childhood and the subject is sometimes referred to as the “child” below and, in other cases, is referred to from the perspective of an analyst as a client.

Based on the principles of learning theory, ABA is an applied science, meaning that practitioners use systematic procedures to teach, resulting in improvements in areas like self-care, communication, academics, behavior reductions, behavior, and/or recreation and leisure. For example, a practitioner may start with 1) choosing a socially relevant behavior, 2) measuring the behavior, 3) using data to determine which treatment to use, 4) using data to implement procedures, and 5) using data to evaluate effects of the treatment. Some of the key characteristics of ABA are:

1. Applied: The chosen behavior is socially important to a person to change his/her daily life. For example, the behavior can be a self-help skill like dressing himself, tying shoes, etc.

2. Behavioral: That specific behavior needs to be improved. Also, the applied behavior analyst can measure that behavior and the measurements are objective and reliable. It is also important for the applied behavior analyst to look at the behavior of all parties involved in the intervention. For example, in some cases, the parents' behavior changes and it leads to a change in the child's target behavior. In this case, the intervention may not be responsible for affecting the target behavior. Similarly, to see the effects of a program to teach children to play with others, an applied behavior analyst may directly observe and measure the interaction between all the children who participate in the program.

3. Analytic: The applied behavior analyst may demonstrate the functional relationship between the behavior and environment. To do this, the applied behavior analyst may control factors in the environment and show that the target behavior has changed because of those factors. For example, the parents may wish to decrease the frequency of the child's tantrum. After observing and taking data, the applied behavior analyst may notice that every time the child throws a tantrum, he gets attention from his parents. Therefore, the analyst may conclude that his parents' attention controls the tantrum behavior.

4. Technological: The procedure that was used in the intervention for the child needs to be described in detail and clearly for others to replicate with different children in the future. That intervention will not be valuable for future reference if no one can replicate it even though it shows its effectiveness for behavior change.

5. Conceptually Systematic: The interventions are based on principles of behavior to explain how and why there is a behavior change. Using conceptual systems enables others to derive further interventions from basic principles and transforms the practice into a discipline.

6. Effective: Effective interventions bring out a behavior change that is socially significant. Such social significance may be analyzed on three levels: the social significance of the goals, the procedures, and the effects. Effective intervention meets the criterion for these three levels.

7. Generality: To be generalized means the behavior change does not stop when the intervention ends. It lasts over time and, more importantly, it appears in different environments other than the ones in which the intervention was implemented. Stated differently, the change can spread to other behaviors besides the target behavior. For example, you teach the child to say “hi” to a person. Later, he says “hi” to someone in the store.

### B. Targets and Programs

The field of ABA, and related fields, use what are called behavioral targets to teach clients new skills. Behavioral targets concern objectively defined behaviors that the practitioner either wants to increase (encourage) or decrease (discourage) in a client. Examples are a practitioner may want to teach a client to vocally say “cow” when presented with a picture of a cow or may want a client to vocally say “milk” when they are thirsty for a glass of milk. They may also want to reduce behaviors, for example, if a client tends to slap themselves when they want attention, the practitioner may want to reduce the number of instances of slapping themselves (ideally to zero), and instead teach the client a “competing” response of saying “excuse me” when they want attention. A behavioral target may include the objective definition of the behavior that is to be increased or decreased, along with the intervention that is to be used to teach it or reduce it.

ABA practitioners currently program for their clients manually: choosing targets that they think are the most appropriate based on assessments, what other targets that client has previously learned, and what they think the client should work on next. Such a program may include a series of targets selected for the client. The selection process is generally based on that client's learning history and goals alone, without substantial attention to the validity of the target itself that is selected. Behavioral targets are then typically manually written by ABA practitioners as needed for a client. The practitioner may manually write the targets from scratch, sometimes copying and pasting from similar targets they have run in the past. Some targets can be reused for multiple clients and may be copied and pasted for this purpose—changing them as needed to adapt to the client or situation.

ABA programming typically is directed to skill acquisition. The priority skills to teach generally focus on the core deficits of Autism: communication, social interaction, and widening narrow interests and activities. The programs selected for instruction should answer the question “What does this individual need to know to be as independent and successful as possible?” The Board-Certified Behavior Analyst (BCBA) develops programs for a client by selecting the most important skills for them to work on, defining targets, and writing instructions for how to implement the targets. Usually, the analyst teaches a target by presenting learning trials and the procedure is run multiple times per day so the child can practice and learn the skill.

Overall, it is the goal of the BCBA to make sure their client is working on meaningful targets, and that they are learning targets quickly, or “mastering” targets (learning a skill to a defined criterion for success, e.g., 3 straight days getting 100% accuracy). They do this by objectively writing targets in a way that they are easy to implement and can be interpreted objectively for success. When trials are run, data is collected to see how well the client is doing, and this data is analyzed by the BCBA to see if the program is effective or needs to be modified.

Traditionally, BCBA's program for clients by writing a comprehensive treatment plan: a comprehensive document outlining what skills the client is working on, how to work on those skills, and providing other information. Treatment plans may be followed by Registered Behavior Technicians (RBTs), who follow the treatment plan and take data on the child's progress. The BCBA can chart the client's progress on each target behavior as shown in FIG. 1 and use a visual analysis of the chart to see if the client is progressing. Software can be used to automate this process. Current software basically recreates what was being done on paper but automates trial presentation for the RBT and makes it easier to organize the data.

### C. Other Data Collection Software in General.

Because ABA is highly data-driven, the software platforms used to organize clients' programs, collect data on their targets, and analyze the data to see how they are progressing, are a critical component to the work. There are a number of data collection programs currently in use in the field. Two of these are DataFinch's Catalyst and Central Reach.

Conventional programs share some common features and it's worth exploring how they implement these functions:

1 Multi-device interaction: Current data collection platforms generally share the characteristic that they are a system of linked devices. BCBA's do their programming typically on a webapp, and the webapp pushes the programming to a data collection iOS app that can be used on a portable device—often an iPad™. These devices sync on different schedules, and a common problem they have is syncing issues, for example, when a problem with the internet makes an RBT lose the data they have been collecting. The programs typically control the iPad to store a few days-worth of data as needed if the internet isn't available to sync the data to their database. For the webapp to update the mobile App, an internet connection is required by both devices.

2 Ability to create and deploy learning trials: Current platforms generally give BCBAs the ability to write their own learning trials, including instructions on how to implement them, and push those trials to the iPad for RBTs to implement. Data is then collected on how the student does on those trials and sent back to the BCBA for analysis and modification as necessary.

3. Ability to collect data during sessions and chart it (both for analysis and reporting).

4. Login for clinicians: Using current platforms, the BCBAs or other clinicians can log in and see their caseloads and programs for clients. This information is HIPAA protected.

### SUMMARY OF THE INVENTION

The present invention relates to a system and associated functionality for developing and implementing programs of Applied Behavioral Analysis for individuals with developmental disabilities. The system allows users, such as BCBAs, RBTs, or other clinicians, to define or access standardized targets that can be readily correlated to those of other clinicians, clients, and programs. The system further enables graphical interfaces for efficient development of programs based on standardized targets and enables improved statistical analysis of collected data. Moreover, the system assists users in identifying appropriate targets for a subject based on the subject's needs and likelihood of success of the target for the subject's program.

In accordance with one aspect of the present invention, a method and apparatus (“utility”) are provided for developing an ABA program for individuals with developmental disabilities. The utility involves developing a data collection, e.g., a database, of defined targets for ABA programs and operating a processing tool having a graphical user interface to manipulate first interface elements, e.g., icons, representing first targets of the defined targets to associate the first targets with a first ABA program. The processing tool may further be used to manipulate second interface elements to define relationships between different ones of the first elements. Each of the defined targets involves a behavior to be addressed and each ABA program is a defined process for modifying one or more behaviors of an individual in relation to one or more of the targets. For example, the defined targets may include behavior goals to be encouraged or discouraged. A user can manipulate the first interface elements by dragging-and-dropping one or more selected icons in relation to a graphical representation of the first ABA program. The second graphical user interface elements may be used to define relationships between targets such as indicating that the targets belong to a group, indicating a parent/child relationship between targets, or defining the location of individual targets with respect to a hierarchy of targets such as defined by a skill tree. In this manner, the user can efficiently generate ABA programs using defined targets that enhance consistency and facilitate statistical processing of results.

In accordance with another aspect of the present invention, a utility is provided for implementing an ABA program. The utility involves: accessing, via a user device, an ABA program appropriate for a subject of interest; selecting a target or “ProbeSet” (a grouping of skills within the system that we want to test if the client is able to do, so we know which ones to teach) of the ABA program; administering the target or ProbeSet to the subject; using the user device to record results; receiving responsive information from a network platform based on the recorded results; and further executing the program based on the responsive information. For example, the recorded results may relate to the results of one or more trials of a target or the results of a ProbeSet. Based on the results, the platform may provide instructions or suggestions concerning one or more additional targets or target parameters. Such instructions or suggestions may be based on analytics implemented by the platform using the results and statistical information derived from analysis of results concerning other subjects, including data from outside companies that can be used to evaluate a subject's performance on targets.

In accordance with a still further aspect of the present invention, a utility provides reports concerning an ABA program for a user, e.g., a responsible party such as a parent or guardian. The utility involves accessing a network platform having subject data concerning results of an ABA program for a subject and for graphical tools. The responsible party can submit a report request concerning the status or progress of a subject with respect to one or more ABA programs. In response, the platform can access the subject data and use the graphical tools to generate a graphical report indicating a status or progress of the subject with respect to the one or more ABA programs. For example, the graphical report may include a skill tree indicating skills achieved and skills remaining to be achieved. The reports provided are especially unique in that they present the clients progress to caregivers in the context of the larger database of all clients working on skills, allowing comparison of how the client is doing compared to expectations.

In accordance with another aspect of the present invention, a utility is provided for assisting users in identifying appropriate targets for a subject based on the subject's needs and the likelihood of success of a target for the subject's program. The utility involves providing predefined targets for ABA programs and a template for structuring entry of target information by a user. For example, the target information may relate to predefined targets, implementation of one or more of the predefined targets, and/or progress towards mastery of the target. The template provides a standardized structure for entry of the target information. In this regard, the template may involve a schema of standardized data fields and values related to the targets, implementation parameters, and criteria for success. The template may be associated with user interfaces including prompts, pulldown menus, icons, or other interface elements to assist a user in accessing and entering information consistent with the standardized structure. The system may then associate the entered target information with metadata identifying data fields, users, subjects, programs, and other parameters.

The utility may be implemented in connection with a network platform that can be accessed by users using user devices such as laptop computers, phones, tablet computers, desktop computers, or other data devices. In many cases, clinicians will access the platform using mobile devices. The utility further involves receiving, at the network platform from multiple users, items of target information for multiple subjects. The items of target information are provided using the predefined targets and a template. A user can then access a collection of target information via the data platform where the collection is based at least in part on the items of information. A user can then enter a query at the network platform concerning an ABA program for a subject. In response to the query, the system can access the collection of target information to generate a response. In order to respect the privacy of subjects and users as well as to comply with any relevant regulations, the response may be free of protected information of other subjects, e.g., personally identifiable information or sensitive medical information. For example, the user can use the system to identify appropriate targets for an ABA program or research effectiveness of targets.

### BRIEF DESCRIPTION OF THE DRAWINGS

For a more complete understanding of the present invention and further advantages thereof, reference is now made to the following detailed description, taken in conjunction with the drawings, in which:

FIG. 1 shows a conventional set of charts for tracking behavior;

FIGS. 2-5 and 10-13 show various user interface screens of a programming application in accordance with the present invention;

FIG. 6 shows an ABA program in accordance with the present invention;

FIG. 7 illustrates a process for developing an ABA program in accordance with the present invention;

FIG. 8 illustrates a process implemented by a user of a program execution application in accordance with the present invention; and

FIG. 9 illustrates a process implemented by system platform in accordance with the present invention.

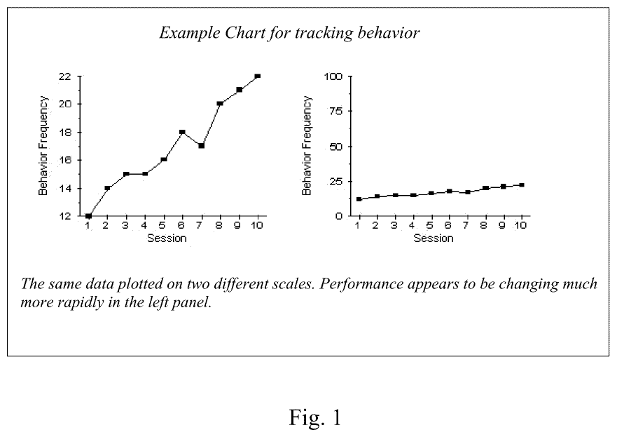

### DETAILED DESCRIPTION

The present invention relates to a system and associated functionality for developing and implementing programs of Applied Behavioral Analysis for individuals having developmental disabilities. The system defines standardized targets, enables graphical interfaces for efficient development of programs based on standardized targets, and enables previously impossible statistical analysis of collected data. In the following description, a specific implementation of such a system is described in detail including examples of user interface screens, targets, and other implementation details. It will be appreciated that the invention is not limited to such implementation details. Accordingly, the following description should be understood as exemplary and not by way of limitation.

The inventive system allows a BCBA to effectively and efficiently program for the children they work with, and for RBTs to implement those programs while working with the children on a remote device. Conventional software requires the BCBA to write out each target for a client. These targets include the behavior to be taught or reduced, the instructions, and other key information that will be discussed later. The BCBA writes each target individually and those targets are synced to the RBT's iPad for them to present as trials. A trial is an instance of running a target. For example, the trial “point to apple” might be run 5 times in a day (5 trials). The app on the iPad instructs the RBT how to conduct the trial, including how many times per day, and the RBT inputs how the student does on the trials (e.g., were they correct or incorrect). This data is synced with a webapp so that the BCBA can see how the student is doing on each target and make changes as necessary.

The limitations with these apps, however, are that targets are each custom written by BCBAs (at best with some parts copied and pasted). This presents two significant limitations:

1 It is a time-consuming process to program for students, as each target must be written out.

2. Very little quantitative analysis is possible on targets because they are all unique. It is difficult or impossible to compare performance across students or targets because there is no standard basis for comparison.

The inventive system diverges strongly from previous ones in key ways: Rather than custom-writing each target, it will have standardized targets drawn on by different BCBAs for programming. Rather than programming via text boxes and writing targets, it will have a graphical interface where BCBAs program for their clients by selecting targets and dragging them to determine the relationships between them. Graphical programming will also introduce the ability to have entirely new ways of visualizing a client's progress through Cumulative Skills Trees—showing each target the client has mastered and the future path of their advancement. Finally, the inventive system has a more user-friendly data collection app that introduces critical improvements over current ones—such as a day-timeline for determining when targets are taught.

1 Standardized Targets: Rather than having BCBAs custom make each target, the inventive program includes a library of standardized targets that they will draw from and implement for their clients. They will be able to modify targets in some minor ways, but in general they will individualize their programs by finding the right target in a library, rather than writing one from scratch. If they can't find the right target, they can choose one that is close and modify it to suit their needs. If the new target passes certain criteria, it will be added to the database of targets so that others can use it in the future. This database of targets will be easily searchable, and it will be easy for the BCBA to add a new target by cloning and modifying a current target that is close to their need but not exact. Further, the targets in the search results will be ranked to aid BCBAs in finding the most effective, commonly used, and applicable targets for their client. By using a database of targets, this system will allow for complicated analyses, including multivariate analyses, across targets, clients, and companies. In particular, this will allow data from outside companies to be used to evaluate a client's performance on targets. This data sharing anchors the decisions a BCBA makes in data from outside sources and gives them an outside frame of reference. This will allow for insights, predictions, and program suggestions based on previous patterns of data across many students—all of which is completely new to the field of ABA.

2. Graphical Programming: Because they are focused on writing new targets for each problem, other data collection software is generally highly text based, where a BCBA spends most of their time writing programs and benchmarks. The inventive system has a graphical user interface where much of the programming is done by dragging highly specific targets from the library into the system interface. New targets can be created when necessary, but smart features of the system will minimize the time spent by BCBA's writing. Where instructions can be copied and pasted between programs by default, they will be. Other components of targets will be standardized to make it very easy to select targets from the library.

3 Cumulative Skills Trees: Because the program allows graphical programming, the progress of clients can be visualized in new and innovative ways—such as a tree showing every skill they have mastered and their future programming to get them to their goals. This will allow BCBAs to see how well-rounded their client's program is, which areas they need help in, and which areas they are advanced in. For example, they may be doing very well in verbal communication, but poorly in independent living skills.

4. ProbeSets: Conventionally, clinicians determine what skills a child needs to learn by using assessments. A BCBA runs an assessment to see what skills a child has mastered and which they need to learn. In our program, assessments (which happen infrequently and are time consuming) will be replaced with ProbeSets. A ProbeSet is skills that a user wants to probe to see if the child knows them. ProbeSets can be implemented easily by simply dropping them into the child's program. ProbeSets will test what skills children have and report the targets that need to be put into their programs to teach. These can be more specific than larger assessments and run much more frequently to guide a child's programming. ProbeSets can be easily customized and reused by BCBA's, making them far more flexible to client needs than assessments.

5. Shared Library of Targets: The inventive system will have a dynamic online database of targets that have been vetted and standardized for ABA practitioners to draw from. The goal of the library is to have a target for nearly any behavior a BCBA could ever want to address. This database will be managed to ensure that targets are useful and non-duplicative.

6. Statistical/Predictive Analysis: Standardization of targets makes possible a valuable component of the inventive system: the ability to use analysis, including data from outside sources, to predict which targets will make learning other targets easier. Statistics will allow the system to determine which targets are most effective, and which methods are most effective. For the first time in the field's history, users will be able to compare methods and techniques on a large scale.

Standardization also enables predictive analysis. Predictive analysis involves predicting how long it will take a client to master a particular target, in the form of how many trials it will take to learn. The system will use variables such as how long it takes other clients to master the target, what skills the client has already mastered, and other factors to predict how long it will take them to learn. This prediction is novel in the field of ABA, where BCBAs use their own guesses to figure how long a client will need to master a target. Predicting how long each target will take to be mastered, and the variables that impact it, will allow BCBA's to arrange conditions in such a way to teach clients much more quickly, such as strategically choosing what targets to teach and when.

Building on this prediction, the inventive system will be driven by a database that has predictive analytics run between all trials to find relationships between which targets make learning other targets easier. These relations will allow the system to suggest the most likely successful targets to help a child learn any target. In addition, the database will be able to take over large portions of programming by drawing on data to decide how to teach a child complex skills more effectively, changing the role of BCBAs forever. This includes using data from outside companies to evaluate a child's performance.

7. Improved Data Collection and Results Reporting: Finally, the inventive system will be intuitive, fun to use, and dynamic. It will implement context-specific trial presentation to make it easier for RBTs to present a variety of trials over the course of the day while still staying organized. It will employ gamification to make teaching fun and emphasize children making progress on trials.

Likewise, a parent portal app will give parents or guardians a window into their child's progress. When their child masters a target, the app will play a notification sound to tell the parent. They will be able to see a snapshot of progress on all domains, and the most important targets currently being worked on. They will be able to navigate into targets and see simplified versions of the instructions RBTs have so that they can implement the targets at home.

### A. System Overview.

The system is divided into multiple parts: a) a webapp where BCBAs organize clients and do their programming; b) a data collection app on mobile devices that sync with the webapp and allow the therapist to implement the programs the BCBA creates; c) a parent portal where parents or guardians can see a snapshot of their child's success and communicate with staff; and d) a webapp for managing the database and analyzing it. All of these may work in connection with a network platform such as a cloud-based platform that executes certain processing as described below.

The BCBA side of the system will allow the BCBA to organize and plan learning targets (concerning skills for the child to learn), which will be presented on the RBT data collection device for the RBT to implement.

In turn, the RBT data collection device guides the RBT on what trials to present, how to do them, how to score them, and collects data on how successful the child was at each trial and sends it back to an analyst portal. This allows the BCBA to adjust the programs to ensure the child is learning as many targets as possible.

The overall goal of the entire system is to allow children, e.g., with Autism, to learn as many meaningful targets as possible, as quickly and thoroughly as possible. For this to happen, targets need to be presented to RBTs in an understandable, fast way. When the child masters targets, new targets need to be implemented to replace them quickly, with no down time while the BCBA analyzes data or chooses new targets. Targets for which a child is not making progress need to be identified quickly, and adjustments made by the BCBA to ensure that time isn't being wasted on programs that aren't helping the child master the skill (maybe this means replacing the target, training the RBT on how to implement it, or just adjusting the target). Each child can be working on as many as 40 targets per day, which are presented in 5-10 trials each. This presents a challenge of ensuring the BCBA can review and address up to 400 trials on a daily basis.

1. Values of System: Below are key values of the inventive system:
(a) Ease of use: the system is intuitive and easy to use. Upon picking it up, a new BCBA will be able to start doing simple programming just by playing with it. More complex tasks can be taught later.
(b) Fun to use: The system makes programming and implementing targets fun. Colors, special user interfaces, and feedback will make it a fun application to use.
(c) Gamification: Making progress with children should be a celebrated and fun activity, and the system harnesses leveling up and skill tree dynamics from video games to tie small successes to overall progress. Think Elden Ring or Skyrim.
(d) Power is in the hands of the user (API, easy export, custom targets, ability to shape database): Other software in the field makes it difficult to download your local data or automate working with it. There are limited APIs, if any. The inventive system is different—it is easy to access your data in excel format, or write an API that accesses your data through another website and uses it in innovative ways. It will have the ability to integrate with other popular applications (Google, Microsoft, etc.).

The inventive system will also enable storing data about targets themselves, across multiple implementations and clients, to better assess their appropriateness for a client. The system stores targets in a database that is shared across providers. Each implementation of each target is stored and uses multiple algorithms to process the targets and help practitioners find the targets that are most likely to help their client learn quickly.

Selecting the most relevant target depends not only on searching for a target that fits the need, but on sorting which targets have the highest probability of success (mastering the skill). Currently, the only way to determine which target will be the most successful is to rely on personal and professional experience to write the target. Following the implementation of the target, the practitioner relies on frequently reviewing the data to see if the target is indeed proceeding towards successful mastery. If it is progressing well, it is left alone. If progress isn't being made, the practitioner makes changes in the hopes of improving its progress. There is no systematic way to analyze a database of targets to determine which one is the most likely to be successful, based on its previous implementations and successes with similar clients. The inventive system involves developing such a database, standardizing the targets to allow analysis and ranking, and helping practitioners select the best target for a client and situation.

The advent of a database of shared targets across ABA practitioners presents the ability to reduce the need to recreate each target for each individual situation, saving time in the process of programming for a client. Targets can be selected for a particular client based on the needs of that client. Rather than writing the target specifically for the client, the practitioner can instead select from the database the target that best fits the client's needs—modifying the target if there doesn't already exist one that is a perfect fit.

The inventive system further involves techniques for analyzing linked databases of targets: individual skills and the behavioral interventions used in Applied Behavior Analysis for teaching them. More particularly, it involves methods used for determining a target's “fit” to a particular client based on the previous targets that client has mastered, and the relationship between that target's mastery and other targets in the database. Further, as mentioned above it predicts how quickly a client will master a target based on their previous performance, targets they have mastered, and multivariate analysis.

The inventive system involves storing targets in a shared database, standardizing targets for analysis, and analyzing the target's performance across implementations to rank them in relation to similar targets. In one implementation, the system provides an objective ranking of targets based on their previous implementation and mastery. The system can also implement a technique for behavioral target ranking in a database for clients who have a large variation in previous target mastery. In addition, a target-ranking method may be implemented that is scalable and can be applied to large datasets of targets such as the database. Additional features of the system will become apparent in view of the description below.

Rather than simply saving and reviewing how a client did on a target/program, and using it simply for that client's future programming, this inventive system stores the information on how the target did with the client. The target is scrubbed of identifying information and stored in a database with other targets that are written in a standardized way. The same target can be run on clients in two different companies, or even states, and will be implementations of the target. This will allow BCBAs to program for their clients in large part by implementing pre-stored targets from the database, rather than writing new targets each time they are programming for a client.

Any practitioner can create new targets in the database as they need them, and the targets they create will be made available to others. To help practitioners find the best fitting targets, the targets created will be analyzed and ranked in searches.

One aspect of the inventive system relates to taking advantage of the stored implementation history of all targets in the database to assign a rank to each target, where the rank is a measure of the “fit” of the target. Rather than determining the fit of the target from its intrinsic qualities (definition, instructions, etc.) or from examination of the data on the progress of the target after implementation, a method consistent with the invention determines quality from the target's history of meeting a uniform objective definition of mastery across previous implementations within the database. Intuitively, a target should be more valuable if when it has been implemented in the past it has successfully met mastery criteria and the client has been able to move on to new targets. Also, targets that have historically been followed by clients learning a larger number of skills are more valuable than targets that have historically not been followed by clients learning more skills. Further, if the target has also successfully met maintenance criteria (the client has retained the skill sometime after the target was initially mastered), that also strongly indicates the quality of the target. Not all masteries are the same, however. The amount of time, or number of trials, that it takes to master a target is a strong indicator of the quality of the target. For two similar targets, if one tends to be mastered in 10 trials and the other in 20 trials, the first target is objectively more valuable to implement with a client, all other things being equal.

Because the probability of mastery within the database predicts the probability of mastery for a particular client, the higher quality behavioral targets are those that have been consistently mastered in the past. Thus, high-quality means that a target is likely to benefit the client, allowing them to master more targets in a shorter period of time by selecting targets that are more quickly and consistently mastered.

As such, the inventive system uses specific algorithms to sort and rank targets stored in the database, based on parameters of their previous implementations, including:
1. The number of times the target has been implemented.
2. How long the target has been in existence since it was written.
3. The ratio of times the target was successfully mastered to times it was implemented.
4. The geographical location of the target (e.g., as a target is mastered more often, it will become more widely available across more practitioners.
5. Statistical characteristics of the target's previous mastery, such as:
   a. Average trials to mastery
   b. Average time (days) to mastery
   c. Standard deviation of the trials to mastery
   d. Standard deviation of the time to mastery
   e. Average number of targets mastered soon after the target was mastered
6. Finally, the relationship between the target and other targets in the database, calculated by comparing their trials to mastery and whether targets have been mastered. For example, in Analyzing Target_{A} the program will look at other targets that the client has mastered and compare the Average Trials to Mastery for Target_{A} across the entire database for instances where Target_{B} was mastered and instances where Target_{B} was not mastered. This will derive a specific relationship (Q) between Target_{A} and Target_{B} c, showing whether having mastered Target_{B} impacts the Trials to Mastery for Target_{A} (will having learned Target_{B} make the client learn Target_{A} quicker). This will show which targets a client should be working on if they want to master a target quickly. It will also greatly improve the programming process by allowing practitioners to rely on actual data about relationships between targets, rather than guessing at relationships.

In the inventive system, these characteristics of targets will allow computer algorithms to greatly improve the selection of targets, by sorting, filtering, and ranking targets based on their appropriateness for a client. In addition to a search algorithm that practitioners can use to find the target they want, results will also be sorted and ranked. For example, targets that have been implemented many times will be ranked higher than targets that have been run a few times. Targets that have been mastered a higher portion of the times they have been implemented will rank higher than targets that have been less successful. Targets will also be ranked by geographic location.

Ranking and sorting won't be just based on the characteristics of a target, but will also be based on the individual client. By assessing the client's learning history, the targets they have mastered and tried to master, the system will use an algorithm similar to that described above to see which targets the client is most likely to learn. It will look at the combination of the Q relationships of all targets they have learned and each potential target to rank potential targets by their likelihood of being learned quickly.

Exemplary algorithms are provided below. Although these algorithms contain specifics for the purpose of illustration, one of ordinary skill in the art will appreciate that many variations and alterations to the following details are within the scope of the invention. Accordingly, the following implementations are set forth without any loss of generality to and without imposing limitations upon, the claimed invention.

TargetRank: Ranks targets/programs by quality.
a. Calculate Mastery Ratio [Ratio of number of Mastery/Implementations] for each target.
b. For each target, rank by higher Mastery Ratios.
c. Search keywords and localization (e.g., within BCBA/company) will outweigh the Mastery Ratio until it becomes higher.
   QRank: Rank targets in search based on previous targets a client has previously mastered.
a. Sum Q for all targets a client has mastered, rank search results for what targets they are most likely to learn quickly, compared to a random Q variable.

### Example Database Management Algorithms:

1. Rank targets by average targets to mastery (T2M).
   a. Calculate Average T2M for total database.
   b. For each target,
      i. Compute an approximation of its Avg T2M to the database T2M.
      ii. If Trial T2M is outside normal range: Flag for review and negatively impact its search ranking.
      iii. Rank targets by approximation to Avg. T2M in database, closer to Avg gives higher rank.
2. For every pair of targets in the database (T₁ & T₂) calculate their P_{TM} (Connection between target success)
   a. What is the impact of having mastered T₁ on the Avg T2M for T₂?
      These algorithms may be implemented in the system as described in more detail below.

### B. System Implementation

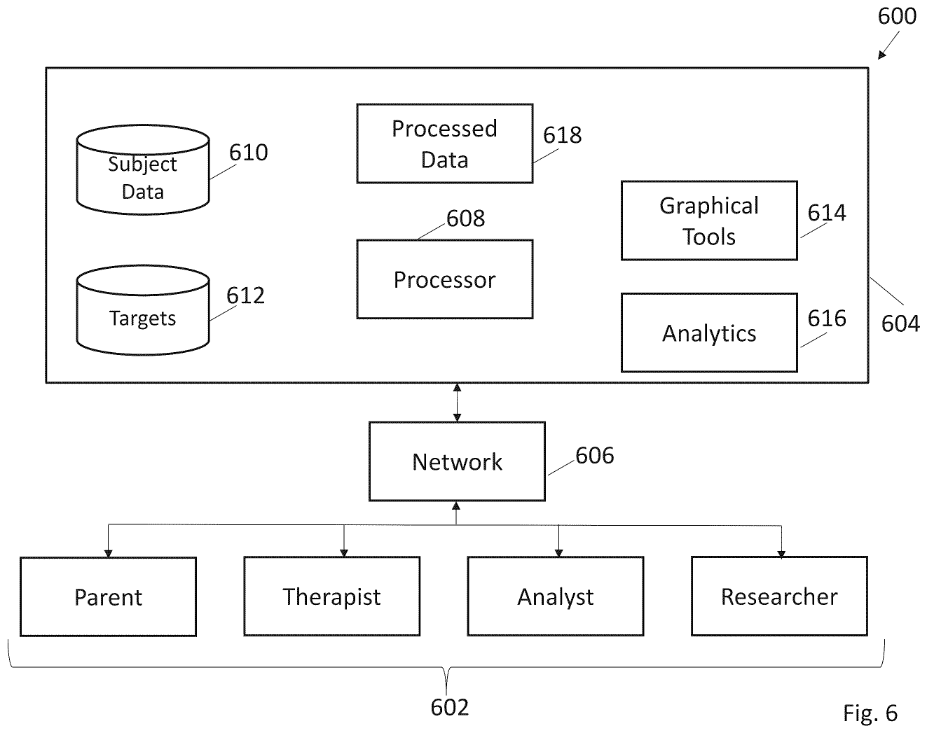

FIG. 6 illustrates an ABA system 600 in accordance with the present invention. The illustrated system 600 generally includes a network platform 604 that can be accessed by various users 602 via a network 606, such as a public or private network, e.g., the Internet. As will be described in more detail below, the system 600 may implement a variety of applications for various use cases. For example, the system can be used to design an ABA program for an individual, to execute a program for such an individual, and/or to perform statistical analyses with respect to the results of programs run on multiple individuals. Accordingly, the system illustrates various potential users 602. Such users may include a parent or other responsible party who can use the system 600 to access information regarding the status of a subject in connection with an ABA program or other information regarding progress. The system 600 may also be used by a therapist in executing an ABA program and recording results of trials, probe sets, and the like. An analyst may use the system 600 to develop programs for individuals and to review execution of the programs as well as results. An analyst or a researcher may use the system 600 to perform statistical analysis or other analyses involving data collected from one or more individuals. Other users are possible in connection with the system 600.

The illustrated users 602 may use a data terminal, either alone or connected to the platform 604 via the network 606. Such data terminals may include, for example, a smart phone or tablet computer, a desktop or laptop computer, or other data network device. The system 600 advantageously enables a variety of graphical programming and graphical data entry functionality as described in more detail below. Accordingly, the user devices preferably include a data device with a graphical user interface system. The user devices may connect to the platform 604 via a wireless and/or wireline network 606.

As will be described in more detail below, the user devices communicate with the platform 604 to implement a variety of functions. Although the platform 604 is illustrated as a single element, it will be appreciated that the platform 604 may be executed on one or more machines (e.g., computers or servers) at a single site or geographically distributed. Each such site may execute the full functionality of the illustrated platform 604 or the functionality may be distributed across sites. Moreover, the functionality may be distributed in various ways between the platform 604, the user devices, and any other platforms, e.g., some preprocessing of information may be executed at the user devices or other platforms, for example, to facilitate rapid response or reduce use of processing resources of the platform 604 or communication bandwidth requirements. The platform 604 may be hosted by a system provider or may be implemented separately (e.g., cloud-based) and connected to the system provider via an interface such as API.

The illustrated platform 604 includes a subject database 610, a target database 612, and other elements, all operated by a processing system including one or more processors 608. The subject database 610 stores information for subjects, e.g., individuals who are the subject of one or more ABA programs such as individuals having developmental disabilities. Such information may include identification information, demographic information, background information such as prior programs executed for the individual and results thereof, program data, result information, and the like. Such information may be accessed by various tools of the system 600, for example, to facilitate implementation of programs, to record results, and to populate various fields in relation to displaying results, for example, in connection with a parent portal.

The target database 612 includes information regarding defined targets. An important aspect of the system 600 is the implementation of ABA programs based on defined and standardized targets. Such targets have been developed by practitioners over time in connection with conventional ABA programs. However, the illustrated system 600 has collected such targets, standardized such targets, defined relationships between complementary or otherwise related targets, and enabled selection of such targets for an ABA program in a graphical programming application. Additional targets may be added to the database 612 as described herein. The information for supporting such functionality may be stored in the target database 612. Such information may include, for each target, an identifier for the target, a description of the target, and various fields, attributes, and values related to the target. The database 612 may be accessed or searched based on these various parameters.

The illustrated platform 604 further includes storage for storing the processed data 618. As described in more detail herein, the system 600 may perform various analyses directed to, among other things: determining effectiveness of various targets or sequences of targets in ABA programs for particular individuals or contexts; predicting what targets or sequences of targets may be most useful for ABA programs for particular individuals or contexts; configuring targets or result information in skill trees or other structures; identifying potential new targets, probe sets, or ABA programs; or the like. Such information and other information may be included in the processed data 618.

The platform 604 also includes logic for implementing a variety of graphical tools 614. As described herein, the system 600 enables graphical programming. For example, an analyst can develop an ABA program by dragging and dropping icons, representing defined targets, into an ABA program under development. Similarly, the analysts can define relationships between individual targets, such as sequencing or grouping, by forming graphical links between the target icons. Other graphical tools 614 facilitate development of skill trees, depictions of status and results, graphical presentation of the results of statistical analyses, and the like. These functions and others are supported by the logic for implementing the graphical tools 614.

The system 600 including the defined targets 612 facilitates aggregation, statistical analysis, and other analysis of data related to targets and programs. Such analysis may be implemented by an analytics module 616. The analytics module 616 may implement algorithms, machine learning logic, and the like for processing subject data, results, and other information to assist in developing ABA programs, implementing ABA programs, and analyzing the results of an individual ABA program or combining information from multiple ABA programs. In this regard, algorithms may be used to calculate information concerning the effectiveness of particular targets, particular sequences of targets, or combinations of targets with contextual information. Machine learning logic such as artificial intelligence may be used to progressively optimize targets, sequences of targets, programs, development of new targets or sequences of targets, and analysis of results. In this regard, a variety of attributes and values may be defined or discovered and this information may be used by the machine learning logic to perform the noted optimization functionality. Such machine learning may be supervised or unsupervised. It is anticipated that the standardized targets 612 as well as the availability of a substantial amount of subject information and results at a centralized platform 604 will enable such machine learning to provide improved targets, ABA programs, and results for subjects.

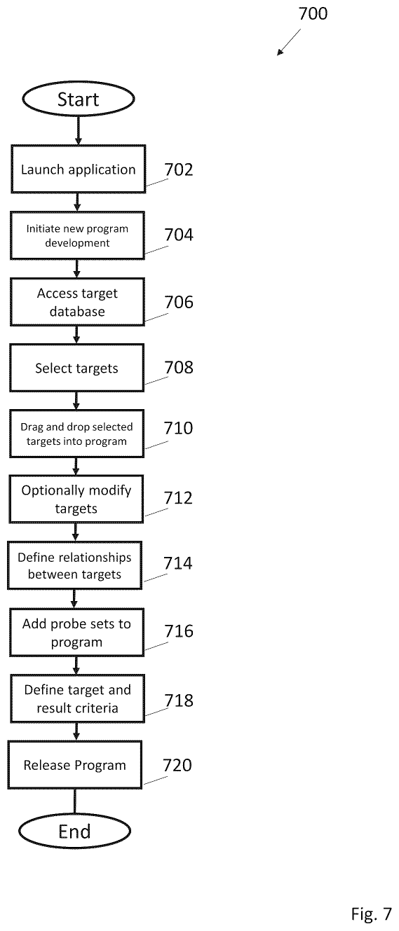

FIG. 7 illustrates a process 700 for use by an analyst, such as a BCBA, or other user in developing an ABA program for a subject. The functionality of the illustrated process 700 (as well as the other processes described below) may be implemented on a user device, a network platform, or distributed across a user device, network platform, and/or other machines. The process 700 is initiated by launching (702) a program development application. Once the application is launched, the user may initiate (704) a new program development project or resume an existing program development project by selecting an appropriate graphical user interface element or otherwise entering appropriate information.

In the inventive system, an ABA program can be constructed using defined and standardized targets. Accordingly, the user may access (706) a target database. For example, the user may be prompted to enter information regarding a desired ABA project or may select a graphical user interface element to access the target database. In response, a screen may be displayed including all available targets or allowing the user to enter information to define relevant targets. For example, such information may relate to defining the nature or severity of the subject's disability, particular behaviors to be encouraged or discouraged, or other information regarding the subject or goals of the program. The format of the screen and prompts may assist in associating the information entered with metadata defining information fields, parameters, and the like, to define a search query to identify appropriate targets. The system may then identify and rank appropriate targets, as described above, using information from the query, statistical information concerning previous results from other analysts/subjects, and the like.

In any event, in the illustrated process 700, the user will receive a graphical user interface including icons representing targets for potential inclusion in an ABA program. The user can then select (708) desired targets and drag-and-drop (710) the selected targets into the ABA program under development. For example, the ABA program may be represented by targets together with links between targets represented by graphical elements as well as other information describing the program, all presented in a panel of a graphical user interface. The targets available for selection may be configured as icons in another panel of a graphical user interface. The user can then add one or more targets to the program by dragging the associated icon or icons from the target panel to the program panel.

The system of the present invention also allows a user to develop custom targets. Such custom targets may be developed from scratch or may be developed by selecting a defined target and modifying (712) the defined target. For example, a user may wish to customize an objective of a target such as changing a predefined target (e.g., “point to apple”) to a customized version of the objective developed for a particular subject (e.g., “point-to-mother”). Various parameters of a target may also be customized such as, for example, frequency of trials, total number of trials, total number of trials per day, context of trials, definitions of success, etc. A process may be provided for approving proposed new or modified targets and adding the targets to the database. Individual targets may be designated as specific to a user or group of users or approved for general use.

The user may also define (714) relationships between targets. For example, multiple targets may be defined as being part of a defined sequence, having a parent or child relationship to one another, being designated as co-requisites for implementation in parallel, being part of a common skill tree or branch of a skill tree, or the like. A variety of graphical representations can be used in this regard such as associating a selected target with a node or sub node of a tree structure, dragging a target to a selected position in a sequence of targets, selecting a defined graphical link for interconnecting related targets, or the like.

The user may also add (716) ProbeSets to an ABA program. As described elsewhere herein, such probe sets can be efficiently implemented in an ABA program without the substantial interruption and time requirements of conventional assessments. Such ProbeSets may be optimized for a program or subject based on the operation of a variety of analytics. Such ProbeSets may be implemented at selected points in a program or may be executed as determined by a therapist and results may be recorded and reported.

In some cases, the defined targets may specify all required criteria related to the target and results. For example, such criteria may relate to the frequency of trials, the total number of trials, the total number of trials per day, and defining success or partial success. In other cases, an analyst or other user may be prompted to define (718) such criteria. For example, the user may be prompted to enter information in a text box or select predefined values related to such criteria from a pulldown menu or other graphical element. Once the user has thereby selected and configured all desired targets, defined all necessary target and result criteria, and otherwise fully defined the program, the project may be released (720) for use in connection with the subject. Such release may be accomplished by selecting a “finish” button or otherwise indicating that the program is completed and the result may be that the ABA program is then made available for selection by a therapist working with the subject.

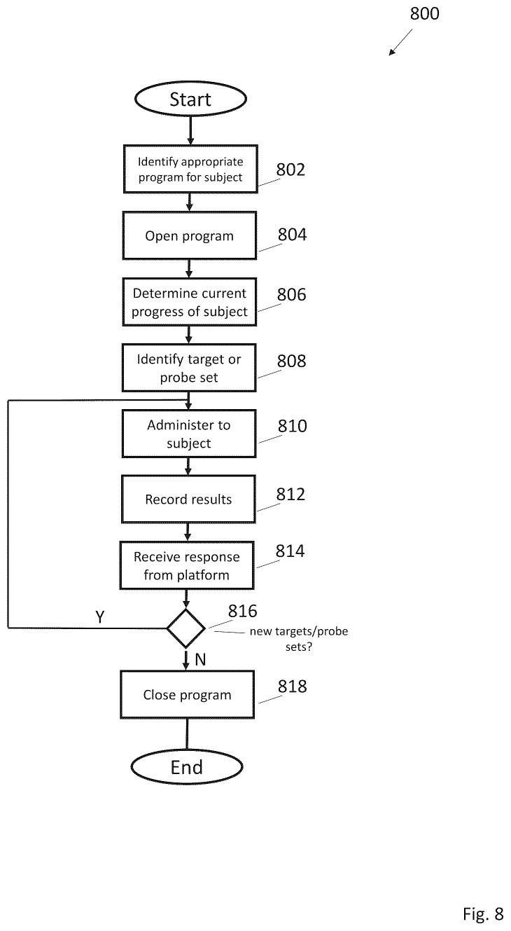

FIG. 8 illustrates a process 800 that may be implemented by a therapist or other user using a program execution application. The illustrated process 800 may be initiated by launching the application and identifying (802) an appropriate ABA program for the subject. For example, the program may be identified using the name of the subject or, if the subject has multiple ABA programs, a specific program may be selected. The program may then be opened (804), e.g., by double-clicking on a corresponding icon of a graphical user interface. Once the program is opened, the user may determine (806) the current progress of a subject. For example, the user may start the program from the beginning, either because it is a new program or the nature of the program is that it is repeated each day or in each session. In other cases, a program may be progressive over multiple sessions and the subject may move on to new targets as prior targets are mastered or completed. In such cases, a graphical representation of the targets of the program may be color-coded, shaded, or may otherwise indicate the current state of progress of the subject in the program.

The user may then identify (808) a target, probe set, or other program element to execute with the subject. For example, a user may select the first target in the program that is not indicated as mastered or completed. The identified target may then be administered (810) to the subject by performing one or more trials of a skill as defined by the target and associated procedures. As the trial or trials are administered, the user may record (812) results. Preferably, at least one part of this recordation involves entering results according to defined criteria relating to success, partial success, or the like. Such defined definitions facilitate objective appraisal as well as subsequent analysis.

As noted above, the process 800 may be executed locally on a client device, executed remotely on a network platform, or the functionality may be distributed across the client device, the network platform, and other platforms. In some implementations, the results may be transmitted to a network platform. The device may then receive (814) a response from the platform. For example, analytics may be implemented at the platform, and the results of the analytics may be used to direct further execution of the program or provide suggestions or other information related to the program. For example, if a result (or series of results) is entered as “partial success,” the platform may suggest modifying certain parameters of the target, based on analysis of data for the subject and/or other subjects, to improve the likelihood of success. In other cases, the platform may suggest alternative targets, rearranging a sequence of targets, or the like. If there are additional targets as part of the program, this process may be repeated (816). Otherwise, the program may be closed (818) and the program data may be updated and saved.

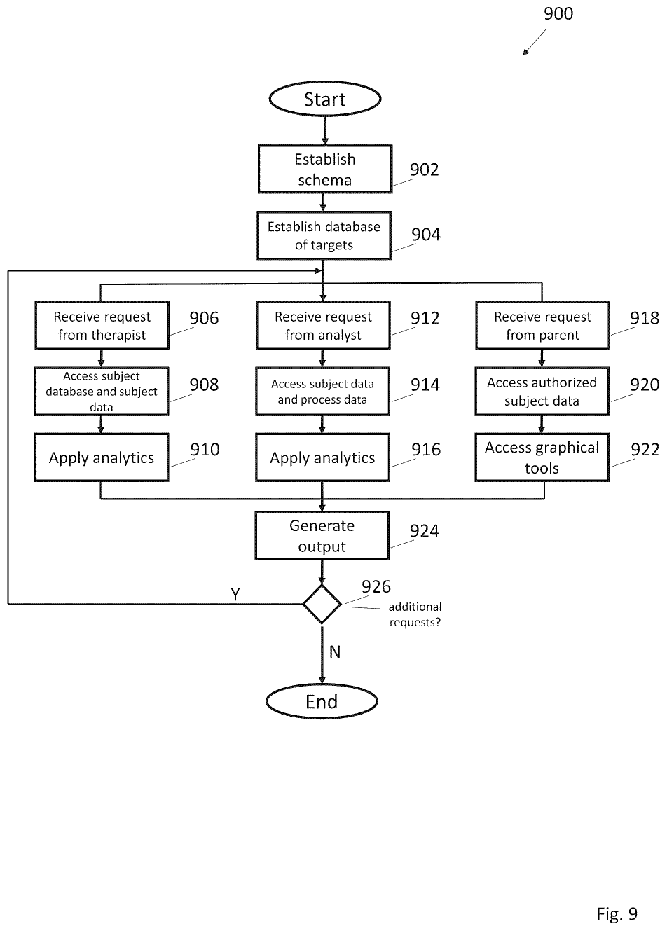

FIG. 9 illustrates a process 900 that may be implemented by a system platform for supporting various applications. The illustrated process 900 is initiated by establishing (902) a schema for the system. The schema may define permissible fields, attributes, and values for data as well as messaging formats and sequences and other data structures for the system. The schema enables efficient and cognizable transfer of information between devices, platforms, applications, and the like. The schema can be used to parse and process data, to index data in databases, to define messaging sequences and protocols for APIs and the like, and other data processing and management purposes. The process 900 further involves establishing (904) a database of targets. As noted above, such targets may be compiled based on analysis of conventional ABA programs and may define a continually evolving set of targets available for selection by users in developing and implementing ABA programs.

The illustrated process 900 involves various processing branches depending on the application or function being executed. In one processing branch, the platform receives (906) a request from a therapist or other user. For example, a request may involve accessing a program for a subject or a request for instructions responsive to results entered during administration of a program. In response to the request, the platform may access (908) the target database, subject data, or other information and analytics. In the case of certain requests, the platform may then apply (910) analytics to determine information responsive to the request. For example, if a therapist or other user enters results of a probe set administered to a subject as part of a program, the platform may then apply analytics to the results of the probe set to implement a predictive analysis. The predictive analysis may identify a proposed target that is determined to be likely to facilitate successful completion of additional targets by the subject based on analysis of subject data or statistical analysis of data relating to other subjects.

In another processing branch, the platform may receive (912) a request from an analyst or other user. For example, the user may be seeking assistance in developing a program for a subject or class of subjects and may wish to obtain statistical information or other processed information to optimize the program. In response to the request, the platform may access (914) subject data and existing processed data. For example, the platform may extract various attributes from the subject data and correlate those to corresponding or related elements of processed data. The platform can then apply (916) analytics to assist in developing the program. For example, the analytics may determine a program, a series of targets, specific target parameters, and corresponding statistical or other information to assist the user in developing the desired program. This may involve comparing the needs of the subject to results of targets implemented by other analysts on other subjects and the results thereof to identify and rank potential targets for the current program.

In a still further branch of the process 900, the platform may receive (918) a request from a parent or other responsible party. For example, a parent may use a parent portal from time-to-time to monitor progress of a subject in relation to one or more programs or objectives. In response, the platform may access (920) authorized subject data. For example, the parent or other user may be required to enter authentication information such as a password or two-factor authentication information. The platform may then identify datasets that the user is authorized to access and the user may select a desired dataset. In response, the user may request specific information such as a status report or skill tree. The platform may then access (922) graphical tools to generate responsive information such as a skill tree identifying the current status or progress of the subject with regard to an associated program or objective.

In connection with any of these processing branches, the platform may then generate (924) an output. In the case of a request from a therapist, the output may include suggestions for executing the current program. An analyst may receive a proposed program, a curated set of targets, statistical information, or the like to assist in developing a program. A parent may receive a skill tree or other status report concerning a subject. In implementing this system, appropriate steps may be taken to ensure that the privacy of subjects, clinicians, or others is respected, and all appropriate regulations are complied with, including, for example, HIPAA (in the United States). In this regard, any sensitive medical information or personally identifiable information may be scrubbed from the data in appropriate contexts, at least prior to generating outputs to third parties. If there are additional requests (926) the process may be repeated. Otherwise, the process may be terminated.

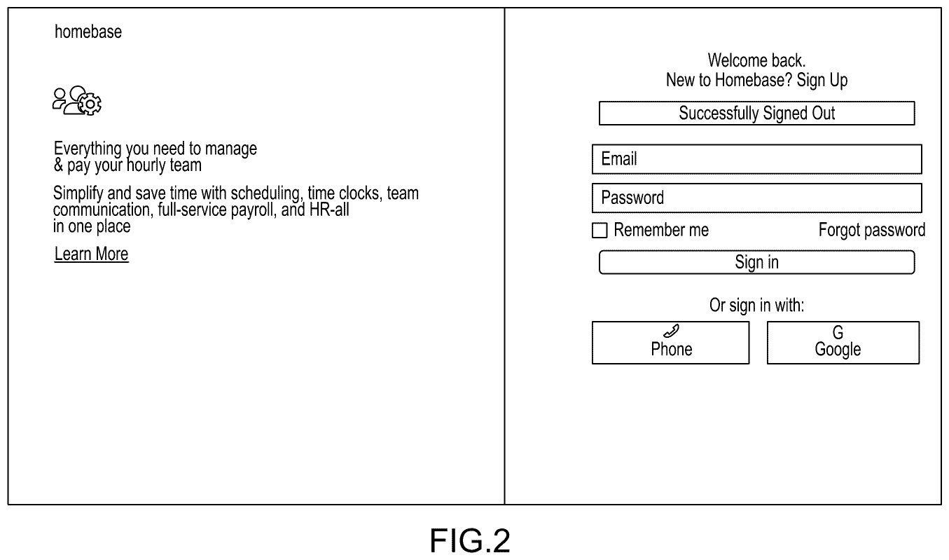

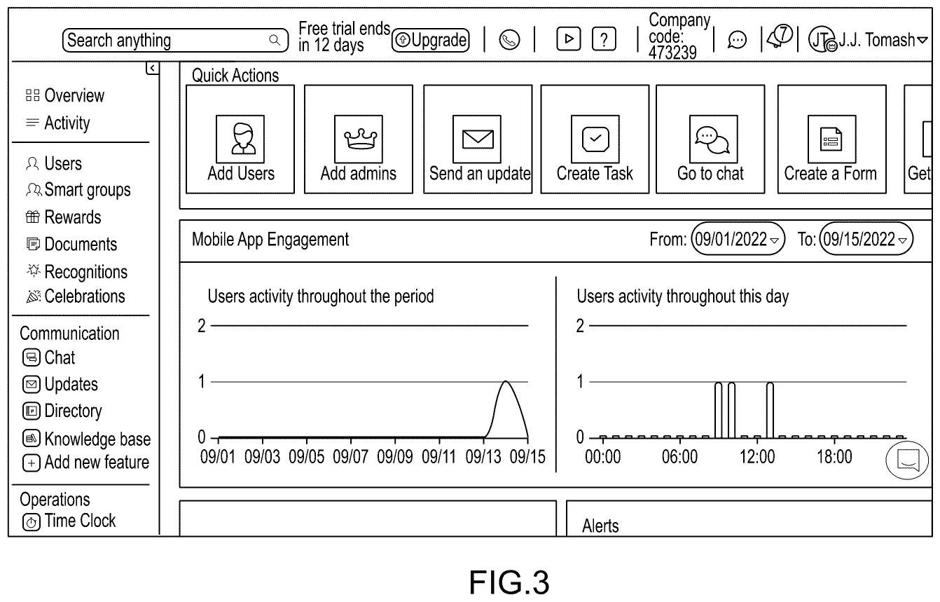

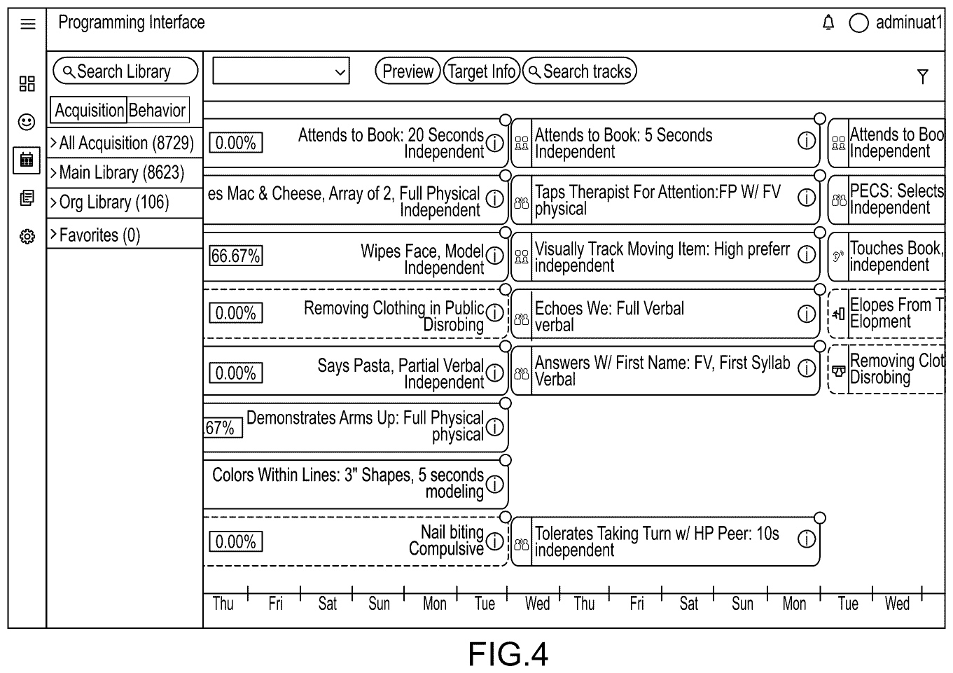

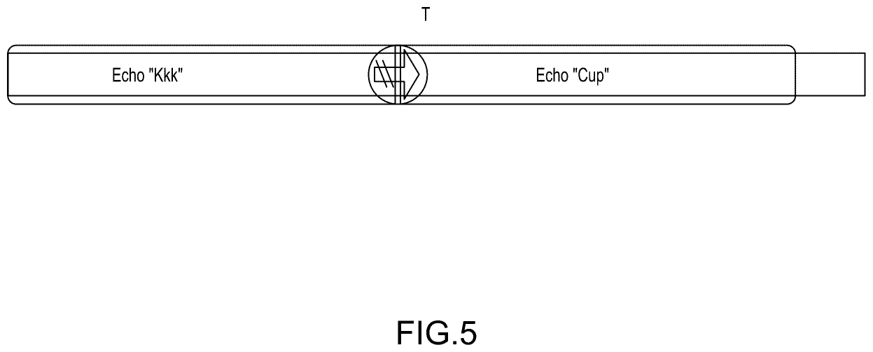

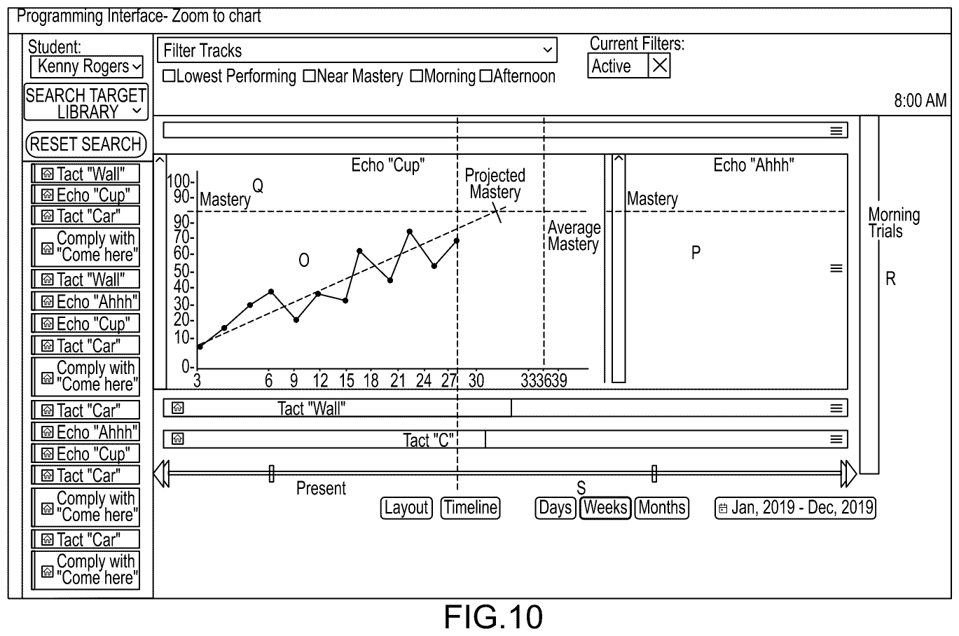

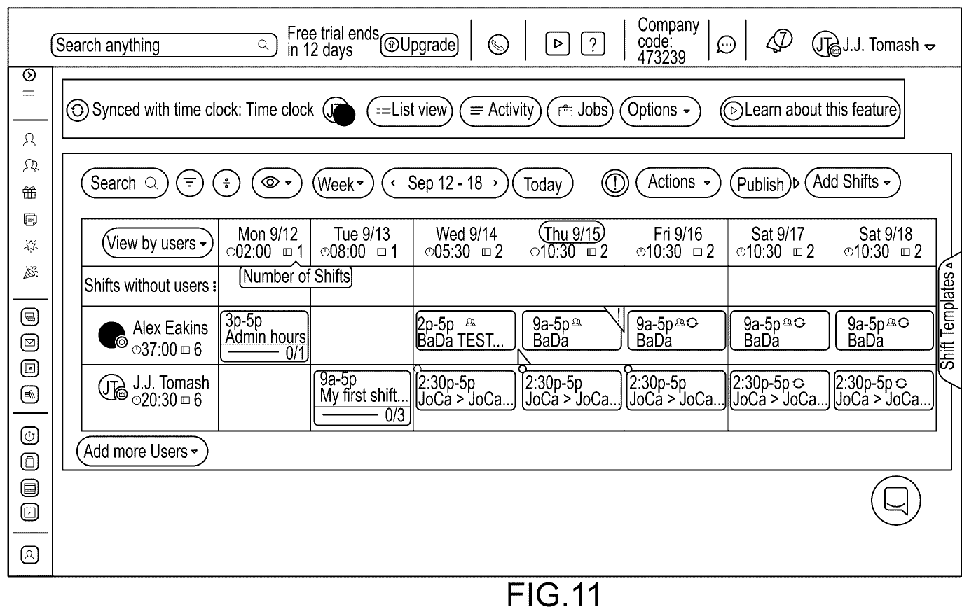

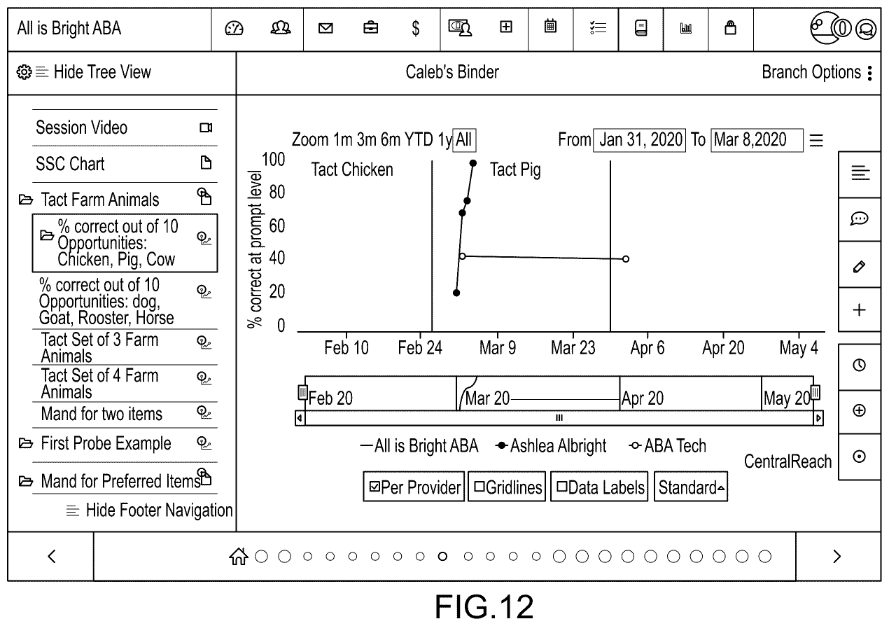

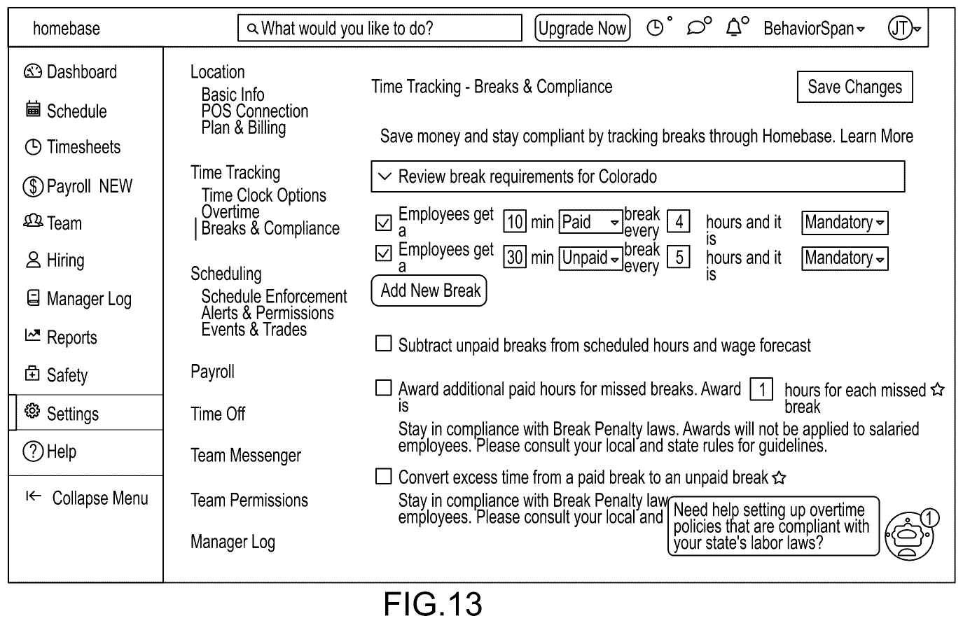

A detailed description of one implementation of the system, executed by an entity or company, follows. Various user interfaces that may be implemented in connection with a corresponding application are shown in FIGS. 2-5 and 10-13.

The system allows a BCBA to create dynamic and complex programs for their students by pulling targets from a standardized database and creating their own targets when the needed one is not available. This program is then pushed to a mobile app that displays trials and instructions for an RBT to implement. These programs will have to be adequate to fill up to an 8-hour day with activities and trials. As the RBT runs trials over the course of the day, the program collects data from the trials and pushes it back to the online database for display on the webapp when the BCBA wants to analyze it and work on the child's program.

Targets are stored in an online database, maintained by the company, that is available to clients of the program. In addition to the information for the target in the library, key data for each target are collected and stored as well. For example, the frequency with which a target is used and trials to mastery will be collected, statistics are run on these online targets to determine relationships between the targets to turn into a predictive network. Analysts at the company will need to be able to run a variety of different analyses on the database and can create new analyses to face new challenges or present new opportunities.

### A. Use Cases.

1. A BCBA wants to see how client is doing on a target. The BCBA goes to the programming screen and zooms in on a target in a learn track by double clicking it. The target is zoomed in on the screen and the graph of current data is displayed within the target.

2. A BCBA wants to export a chart for reporting. The BCBA goes to the analysis and reporting page and finds the desired target in the sidebar (searchable or navigable). They drag the target to the chart area to display the data, phase change lines, and trend on the chart. To compare other charts, they can drag more targets to the graphing area. They can select a different student and add their targets to the graph as well. They can remove targets by clicking the “X” on the list of displayed targets. They can also scale the X and Y axes as needed and change the title and axis titles. When BCBA has the chart as they want it, they click a button and the system either downloads the image or saves it to the clipboard to easily paste into a document.

3. A user wants to export data. The user navigates to the analysis and reporting page and clicks “Export Data” in the bottom corner. The system presents a confirmation page and a link to download the data from all current and past targets for the client as a CSV.

### B. Database of Targets.

1. Standardized Targets, with Adaptability for different clinics: In this implementation, each target in the database contains the following attributes:
(a) Target Name: name of the target
(b) Target Number (internal use)
(c) Target Type:
(d) Data Type:
(e) Goal Name (for reporting): how it will be reported on chart
(f) Skill Area:
(g) Tags:
(h) SD1:
(i) SD2:
(j) SD3:
(k) Behavior Definition
(l) Error Correction
(m) Default/Suggested Mastery Criteria
(n) Creator:
(o) Target SD Instructions: (partially internal organization)
(p) Correct Response Instructions (internal organization)
(q) Error Correction Instructions (internal organization)
(r) Tags (internal organization)
(s) Number of Daily Trials (internal organizations)
(t) Mastery Criteria (internal organization/user settable)
(u) Context (internal organization)
(v) Average Trials to Mastery
(w) Number of implementations
(x) Percentage mastery/implementation
(y) Default/Suggested Mastery Criteria

1. Target Name: name of the target (important that it is specific enough that there are no duplicates)

2. Target Number (internal use): Used for program purposes

3. Target Type: Needs work for bigger categories (Free Operant vs DTT, Accel vs. Decel, etc)
(a) Anecdotal
(b) Conditional Discrimination
(c) DTT *
(d) Duration—duration of instance of behavior
(e) Echoic—echoing a sound or word back
(f) Fluency/Rate
(g) Frequency—number of times behavior occurs
(h) Grouped Frequency
(i) Jump-To
(j) Quantity
(k) Task Analysis *
   (1) Prompt Levels:
      i. Independent
      ii. Model
      iii. Physical
      iv. Verbal
      V. Gestural
      vi. Positional
      vii. No Response
      viii. Correct (optional)
      ix. Incorrect (optional)
(l) Toileting
(m) Whole/Partial Interval
(n) Decel Behavior: targets for behaviors that we want to decrease.

3. Test Target Types:
(a) Free Operant:
   (1) Decel:
      i. Duration—duration of instance of behavior
      ii. Frequency
      iii. Magnitude
      iv. Whole/Partial Interval
      v. Other
   (2) Accel:
      i. Duration—duration of instance of behavior
      ii. Frequency
      iii. Magnitude
      iv. Whole/Partial Interval
      v. Other
(b) Contrived:
   (1) Conditional Discrimination
      i. DTT
      ii. Task Analysis
      iii. Fluency/Rate
      iv. Toileting
         Free Operant
         Toilet Trials

4. Goal Name: How the target will be displayed on charts

5. Skill Area:
(a) Attending
(b) Communication/Mands
(c) Communication/PECS
(d) Communication/SGD
(e) Daily Living Skills
(f) Echoics
(g) Feeding
(h) Imitation
(i) Intraverbal
(j) Listener Responding
(k) Motor Skills
(l) Parent Training
(m) Play Skills
(n) Social Skills
(o) Tact
(p) Toilet Training
(q) Toleration
(r) VPMTS

6. Tags: Tags that are associated with the target to make it easily findable in a search or browsing.

7. SD: The stimulus presented by the RBT immediately prior to the behavior being expected.

8. Behavior Definition: Objective description of the target behavior, which can be agreed upon by different observers.

9. Creator: The person/organization that created the target.

10. Date Created: The date that the target was validated into the target database

11. Target SD Instructions:

12. Correct Response Instructions:

13. Error Correction Instructions (if applicable):

### Internal Organization Editable Fields:

14. Tags (internal organization): Tags that a client can put on targets to help them find it.

15. Number of Daily Trials (internal organizations): Number of times a trial will be presented each day.

16. Mastery Criteria (internal organization/user settable): Criterion student needs to meet for target to be mastered and learning track to progress to new target.

17. Context (internal organization): Time of day/activity trials occurs in

### C. Basic Target Program Template & Criteria

| Skill Acquisition | | | |
|---|---|---|---|
| Formatting | | Example without Benchmarks | Example with Benchmarks |
| Target Name | | Tolerates Waiting 30 Seconds | Tolerates Waiting |
| | | w/ Visual Timer | |
| SD 1 | | “Let's wait” + presentation | “Let's wait” + presentation |
| SD 2 | | of visual timer | of visual timer |
| SD 3 | | | B1: Waits for 1 second |
| | | | Jul. 29, 2022 |
| | | | B2: Waits for 5 seconds. |
| | | | Aug. 3, 2022 |
| | | | B3: Waits for 15 seconds |
| | | | Aug. 15, 2022 |
| | | | B4: Waits for 30 seconds |
| | | | Aug. 20, 2022 |
| Procedure Steps | 1. | Contrive a situation in which | Contrive a situation in |
| | | the client could gain access | which the client could gain |
| | | to a high preferred | access to a high preferred |
| | | reinforcer. | reinforcer. |
| | 2. | Before giving them the | Before giving them the |
| | | reinforcer present the SD. | reinforcer present the SD. |
| Correct Response | | Client waits for 30 seconds | Client waits for benchmark |
| If needed, specific | | before receiving the | specific period of time and |
| reinforcers can be specified | | reinforcer and does not | does not engage in |
| in this section. | | engage in maladaptive | maladaptive behaviors. |
| | | behaviors. | |
| *Error Correction | 1. | Mark as incorrect | Mark as incorrect |
| Steps if client engages | 2. | Redirect to another activity. | Redirect to another activity. |
| in behaviors . . . | 3. | Wait until they are happy, | Wait until they are happy, |
| | | relaxed, and engaged for 1 | relaxed, and engaged for 1 |
| | | minutes. | minutes. |
| | 4. | Re-present trial at 15 seconds | Re-present trial at the |
| | | | previous benchmark. |
| | 5. | If the client still engages in | If client still engages in |
| | | behaviors move on and try | behaviors move on and try |
| | | trial again at a later time of | trial again at a later time of |
| | | day | day. |

| | |
|---|---|
| *Additional Notes | Only run during table time, |
| (Optional) | not during play w/ peers as |
| | this could cause dangerous |
| | behaviors towards peers. |

Goal Progession: As seen currently as benchmarks. The default setting is moving to
continuing benchmarks after 2 consecutive data points at 100%.
Final Goals: Default setting for goals is that they master out after 5 consecutive data points
at 100%.
Passed Baseline: These close out immediately but can be reopened in the event of a fluke
trial or regression in skills.
Maintenance:
* After a final goal has reached 100% for 5 consecutive days it moves to weekly
maintenance, then semi-weekly, then monthly. After monthly the target is closed.
Each maintenance program is only run w/ 1 trial.
If a program fails a trial in maintenance, it reverts back one stage. (ex. Failed weekly
goes back into programming, failed semi-monthly goes back to weekly.)
If a maintenance program goes back into daily programming, it has to achieve 5
consecutive data points at 100% again before mastering out.
Why 5?
We use 5 trials to provide enough practice for the skill and to show stable
responding. For clients who cannot tolerate that many trials (ex. as in a toleration program
or daily living skills program), we drop them but try to fade back to 5 when possible.
Catalyst makes us select a number that is consistent across all kids, but we change
that number depending on the client's ability and challenges (ex. practicing hand washing 5
times a day may be overkill for some kids or practicing toleration programs only 5 times a
day for kids who learn from higher learning trials is too little).
Some say 10 trials is a good criterion for effective learning
(https://files.eric.ed.gov/fulltext/EJ846778.pdf), but we found that we had clients who would
master and retain information so quickly that we would only need 3 data points at 100%.
Usually if a skill is dwindling or failing maintenance more than once, we increase the trials
provided and the number of days till mastery.
We also use 100% mastery because emerging evidence suggests that higher
mastery criteria result in more effective skill maintenance
(https://onlinelibrary.wiley.com/doi/full/10.1002/bin.1778).

| | |
|---|---|
| Prompt Codes: | Independent |
| | Model |
| | Physical |
| | Verbal |
| | Gestural |
| | Positional |
| | No Response |
| | Correct (optional) |
| | Incorrect (optional) |

Further prompt codes should not be added to the list and should instead be defined
as a progressing goal (ex. Claps Hands Using Partial Physical Prompt).

| Target Types Included w/ Catalyst: | Anecdotal Conditional Discrimination | |
|---|---|---|
| (* are ones we use most) | DTT * | |
| | Duration * | |
| | Echoic | |
| | Fluency/Rate | |
| | Frequency * | |
| | Grouped Frequency | |
| | Jump-To | |
| | Quantity | |
| | Task Analysis * | |
| | Toileting | |
| | Whole/Partial Interval | |
| Skill Areas: | Attending | |
| | Communication/Mands | |
| | Communication/PECS | |
| | Communication/SGD | |
| | Daily Living Skills | |
| | Echoics | |
| | Feeding | |
| | Imitation | |
| | Intraverbal | |
| | Listener Responding | |
| | Motor Skills | |
| | Parent Training | |
| | Play Skills | |
| | Social Skills | |
| | Tact | |
| | Toilet Training | |
| | Toleration | |
| | VPMTS | |
| Behaviors for Decrease | | Example |
| * Definition | | Screams (Isolated Instances): Any instance of client |
| | | raising his voice above conversational volume making |
| | | an “ah” sound each instance is separated with 5 |
| | | seconds of non-screaming voice. |
| * Examples & Non-Examples | | Example: Echoes “go” followed with screams; client |
| | | talks in normal voice. |
| | | Non-Example: Screams while playing or watching |
| | | videos. |
| | | Note: If screaming occurs more than 3 times without |
| | | pause, start crying/screaming duration timer. |
| * Procedure | 1. | Prompt functional communication (ex. “what can we |
| * May be broken up by | | say instead, “tell me” + presentation of SGD device, |
| antecedent type. | | etc.) |
| | 2. | If the client responds correctly, honor his mand. |
| | 3. | If he continues screaming, keep a safe distance without |
| | | disengaging (continue providing verbal prompts.) |
| | 4. | Wait until calm for 15 seconds to re-present the verbal |
| | | mand, |
| | 5. | If client doesn't respond after 2nd presentation, |
| | | reinforce bait and prompt FCT. |
| Denied Access: | 1. | Ignore behavior and say, “not available”. |
| | 2. | If client does not redirect himself after 30 seconds |
| | | reinforcer bait |
| Demand Placed: | 1. | Ignore behavior and prompt functional communication |
| | | via PECS, SGD, Sign, or Verbal Mands. (Ex. “you can |
| | | say no”) |
| | 2. | If he responds, allow him space but deny access to |
| | | reinforcers. |

| | |
|---|---|
| Note: | If he screams while imitating/manding response do not |
| | honor and prompt functional communication again. |

### D. Categorization and Searching.

The database will be massive, and it is important that it is easy for BCBAs to find the target they are looking for easily. Them being able to find the right target easier than they can write a new target is critical to the program being usable. For this reason, they will be able to navigate the library in different ways: categorization/browsing and smart searching. To help further, a smart prioritization will display first the targets that are most likely the ones that they want.
1. Different Levels of categorization:
2. Smart search:
3. Prioritization functionality:
   E. Predictive analysis and functionality
   F. Modeling of programs through analysis of previous programs
   G. How Shared Database is managed by system operator, and independent libraries for clinics and clients.
1. Multi-tenant architecture
2. Data syncing
3. Requirements for individual targets to be assimilated.
4. Auditing of targets in Database, requirements for clinic-specific targets

H. How targets are implemented and rendered into Data Collection app

I. ProbeSets and targets as trials or probes.

J. Inclusion materials in libraries (e.g., tact cards), variation of stimuli during trials, etc.

K. Data analysis of performance on targets, prioritization, and eventually predictive network of targets. A core function of this program is the ability to run data analysis on the targets in the library to look for patterns. For example, across different subjects that master a target, is there another target or combination of targets that predict that they will learn it quicker? Multivariate analysis across the entire database will look for patterns in the data and use that to improve the database and add functionality.

1 Data collected:
(a) Trials to mastery: Across subjects, how many trials it takes to master a target.
(b) Frequency of implementation: How often is a target implemented in programming across different students, organizations.
(c) Correlations with other targets (targets it tends to be implemented after, before, at the same time as): what patterns are there in a target being implemented compared to other targets. Are there any other targets strongly correlated?

### 2 Types of Analysis to be Run:

(a) Target Optimization: Looking for targets that need improvement or to be removed. Targets that have been in the database for a long time but have never been implemented need to be addressed. Either their ability to show up in searches needs to be improved, they need to be modified to make them more usable, or they need to be deleted. Likewise, targets that are implemented, but are not being mastered quickly are ineffective, and need to be modified to improve their performance. Perhaps a target needs to be broken into two targets, the instructions need to be improved, or the mastery criteria is too high.
(b) Normal Distribution: In theory, targets in the database should have a normal distribution in terms of the number of trials required to mastery. Targets that exist in the tales of the distribution, meaning it takes too many or too few trials for them to be mastered, need to be addressed by Database Moderators.
(c) Internal Target characteristics: Each target will have its own internal characteristics in the data. For example, the average trials to mastery for a target would be a useful piece of information to display to BCBAs looking to program. It can show them if their student is learning slower than the average, or faster than the average. It would also help them to plan out how long it should take for clients to master targets. Other internal characteristics would also be valuable: most common alterations made when implemented; average mastery criteria used; how often a target is spliced during implementation; what targets are usually spliced into it during implementation?
(d) Automatic Search Optimization and Categorization: Online retailers do not manually categorize every single product on their website. They typically use an algorithm that looks at search patterns and implementations to tag products and create categories for them for future searches. The same is true for the database of the inventive system. It will continuously improve itself based on searches and categorizations, giving itself new search terms, labels, and categories depending on how users find it.

ProbeSet: A set of targets that, when implemented, probes whether the student has mastered each target. Upon completion, program returns unmastered targets as suggestions for programming. If none of the targets are mastered after a certain number of trials, the ProbeSet is aborted and the program suggests prerequisite targets and ProbeSets. ProbeSets can be created either in the Program Library, selecting a Standard ProbeSet, or by selecting one or more targets on the graphical display (dragging selection area around them) and clicking “Create ProbeSet.”

GenSet: A set of targets that, when implemented, trains a student to master a target across different settings and therapists. If none of the targets are mastered after a certain number of trials, the GenSet is aborted, and the program suggests prerequisite targets and ProbeSets. Targets that pass generalization repeatedly are shown as larger on visual programming display. GenSets can be created by selecting one or more targets on the graphical display (dragging selection area around them) and clicking “Create GenSet.”

MainSet: A set of targets that, when implemented, probes maintenance of targets over time after mastery. If any target fails mastery after a certain number of trials, program returns it as suggestions for programming. Targets that pass mastery repeatedly are shown as darker on visual programming display. MainSets can be created by selecting one or more targets on the graphical display (dragging selection area around them) and clicking “Create MainSet.”

SkillSet: A set of targets that has been mastered. Displayed on the graphical programming interface as a module with solid lines going to each target.

Standardized Target: A target that is synced with the Database and available for users in different organizations.

Non-Standardized Target: A target that has been cloned and changed, and hence is not available in the Database and does not have all features of a Standardized Target (such as analytics and prediction). Non-standardized attributes of the target that have been modified from the Standard are highlighted yellow. Once Assessed, Target may be added to the Database and Standardized.

Flexible Attributes: Attributes of a Target that can be changed within an organization without cloning a target. For example, instructions on targets are flexible.

Fixed Attributes: Attributes of a Target that cannot be changed within an organization without cloning a target. For example, Sd on targets are fixed.

Cloning a Target: Making a copy of an existing target to change attributes for programming. This allows a user to implement a more “custom” target by changing normally fixed attributes. The cloned target is non-standard until it has been reviewed and assimilated into the database as a new target.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

What is claimed:

1. A method for use in developing and implementing programs of Applied Behavior Analysis (ABA) for individuals with developmental disabilities, comprising:
providing predefined targets for ABA programs, wherein each said predefined target comprises a skill to be addressed and one or more interventions for promoting mastery of said skill, and each said ABA program is a defined process for modifying one or more behaviors of an individual in relation to one or more of said predefined targets;
providing a template for structuring entry of target information by a user concerning one or more of said predefined targets, implementation of said one or more of predefined targets for a subject, and progress towards mastery of one of said predefined targets, wherein said template provides a standardized structure for said entry of target information;
receiving, at a network platform from multiple users, items of target information for multiple subjects, said items of target information provided using said predefined targets and said template;
establishing a collection of said target information, accessible via said network platform, based at least in part on said items of target information from said multiple users;
receiving, at said network platform, a query from a first user concerning a first ABA program for a first subject; and
accessing said collection of said target information to generate a response to said query, said response free of protected information of other subjects.

2. The method of claim 1, wherein said skill involves reducing a behavior associated with a developmental disability.

3. The method of claim 1, wherein said skill is associated with learning a behavior that is suppressed due to a developmental disability.

4. The method of claim 1, wherein said query concerns identifying an appropriate target for said first subject.

5. The method of claim 4, wherein said network platform is operative for identifying a first behavior based on said query and matching said first behavior to one or more candidate targets.

6. The method of claim 5, wherein said matching comprises identifying a plurality of said one or more candidate targets associated with said first behavior and comparing said plurality of said one or more candidate targets based on a likelihood of success in promoting mastery of an associated skill.

7. The method of claim 6, wherein said network platform is operative for ranking said one or more candidate targets based on one or more of a number of times each of said candidate targets has been implemented, how long each of said one or more candidate targets has been in existence, a ratio of the number of times each of said one or more candidate targets was successfully mastered to times it was implemented, a geographical extent over which each of said one or more candidate targets was implemented, and statistical characteristics concerning previous mastery of each of said one or more candidate targets.

8. The method of claim 6, wherein said network platform is operative for evaluating said likelihood of success for a first candidate target by considering a relationship between mastery of said first candidate target and mastery of another target.

9. The method of claim 1, wherein said network platform is operative for receiving information from one of said multiple users for defining a new target and making said new target available to others of said multiple users.

10. The method of claim 1, wherein said network platform is operative for receiving, in said standardized structure, result information for an implemented target.

11. The method of claim 1, wherein said network platform is operative for predicting how long it will take a first subject to master a skill.

12. A system for use in developing and implementing programs of Applied Behavior Analysis (ABA) for individuals with developmental disabilities, comprising:
a network platform operative for:
providing predefined targets for said ABA programs, wherein each said predefined target comprises a skill to be addressed and one or more interventions for promoting mastery of said skill, and each said ABA program is a defined process for modifying one or more behaviors of an individual in relation to one or more of said predefined targets;
providing a template for structuring entry of target information by a user concerning said one or more predefined targets, implementation of said one or more predefined targets for a subject, and progress towards mastery of said one or more predefined targets, wherein said template provides a standardized structure for said entry of target information;
receiving items of said target information for multiple subjects, said items of target information provided using said one or more predefined targets and said template;
providing access to a collection of said target information, said target information based at least in part on said items of target information from said multiple subjects;
receiving a query from a first user concerning a first ABA program for a first subject; and
accessing said collection of said target information to generate a response to said query, said response free of protected information of other subjects.

13. The system of claim 12, wherein said skill involves reducing a behavior associated with a developmental disability.

14. The system of claim 12, wherein said skill is associated with learning a behavior that is suppressed due to a developmental disability.

15. The system of claim 12, wherein said query concerns identifying an appropriate target for said first subject.

16. The system of claim 15, wherein said network platform is operative for identifying a first behavior based on said query and matching said first behavior to one or more candidate targets.

17. The system of claim 16, wherein said matching comprises identifying a plurality of said one or more candidate targets associated with said first behavior and comparing said plurality of said one or more candidate targets based on a likelihood of success in promoting mastery of an associated skill.

18. The system of claim 17, wherein said network platform is operative for ranking said plurality of one or more candidate targets based on one or more of a number of times each of said plurality of one or more candidate targets has been implemented, how long each of said plurality of one or more candidate targets has been in existence, a ratio of the number of times each of said plurality of one or more candidate targets was successfully mastered to times it was implemented, a geographical extent over which each of said plurality of one or more candidate targets was implemented, and statistical characteristics concerning previous mastery of each of said plurality of one or more candidate targets.

19. The system of claim 17, wherein said network platform is operative for evaluating said likelihood of success for a first candidate target by considering a relationship between mastery of said first candidate target and mastery of another target.

20. The system of claim 12, wherein said network platform is operative for receiving said items of target information from one of said multiple subjects for defining a new target and making said new target available to others of said multiple subjects.

21. The system of claim 12, wherein said network platform is operative for receiving, in said standardized structure, result information for an implemented target.

22.-56. (canceled)
